(19) United States
(12) Patent Application Publication
(10) Pub. No.: US 2024/0041790 A1
(43) Pub. Date: Feb. 8, 2024

(54) TOPICAL ANTISEPTIC

(71) Applicant: SurgiWipe LLC, Philadelphia, PA (US)

(72) Inventors: Michael YAYAC, Philadelphia, PA (US); JAVAD PARVIZI, Philadelphia, PA (US); HARRY BRITTAIN, Philadelphia, PA (US); JEONGEUN CHO, Philadelphia, PA (US); Aradhna SAXENA, Philadelphia, PA (US); MAYANK PARMAR, Philadelphia, PA (US)

(21) Appl. No.: 18/382,741

(22) Filed: Oct. 23, 2023

Related U.S. Application Data
(63) Continuation of application No. PCT/US2022/025995, filed on Apr. 22, 2022.
(60) Provisional application No. 63/178,055, filed on Apr. 22, 2021.

(51) Int. Cl. A61K 9/70; A61K 31/191; A61K 9/107; A61K 31/455; A61K 36/61; A61K 47/10

(52) U.S. Cl. CPC A61K 9/70; A61K 31/191; A61K 9/107; A61K 31/455; A61K 36/61; A61K 47/10

(57) ABSTRACT

An antiseptic composition for penetrating and treating the dermis and/or pilosebaceous follicle of a mammal, said antiseptic composing comprising: (a) a sebostatic agent for reducing sebum production to prevent clogging of skin pores; (b) a barrier-forming emollient for forming a protective barrier film to aid said dermis in maintaining moisture and to shield said dermis from the environment; and (c) an antiseptic agent.

### REFERENCE TO RELATED APPLICATION

This application is based on U.S. Provisional Application No. 63/178,055, filed Apr. 22, 2021, which is hereby incorporated by reference.

### FIELD OF INVENTION

This invention relates generally to topical antiseptic solutions used to reduce microorganisms on the skin that may cause infection, and, more specifically, to topical antiseptics that penetrate the dermis and treat the dermis.

### BACKGROUND OF INVENTION

Preoperative skin preparation with an antiseptic agent is a key measure in prevention of surgical site infection (SSI). Use of antiseptic skin preparation product immediately prior to draping and incision has become standard of care and current SSI prevention guidelines additionally recommend further cleansing of the skin with soap or an antiseptic agent on at least the night prior to surgery.

Current antiseptic products rely on a single or combination of fast-acting broad-spectrum agents, such as chlorhexidine gluconate (CHG), iodine complexes, and isopropyl alcohol (IPA), to reduce gram-positive and gram-negative bacteria as well as fungi that reside on the skin. Examples include 2% CHG/70% IPA and Iodine Povacrylex[0.7% available iodine]/74% IPA which are used immediately prior to incision. Products including 4% CHG soap, 2% CHG presaturated cloths, and 5-10% povidone-iodine (PVP-I) solutions are often used to decolonize the skin and nares as part of preoperative or intraoperative preparation. These agents act through nonspecific mechanisms such as protein denaturation and microbial cell membrane disruption making them very effective against a wide array of microbial species.

One issue with a single active antiseptic agent is that microbial resistance can emerge. For example, in recent years, Staphylococcus aureus has been shown to be resistant to CHG. Despite the presence of numerous antiseptic solutions and wipes and other infection prevention strategies, SSIs still occur at an incidence of 2-5% and microorganisms present on the patient's skin are still cited as a major source of these infections.

While the antiseptic agents used are known to be both potent and broad, complete eradication of all organisms from the skin is unlikely to be achieved for a number of reasons. For example, there are around 1,000,000 organisms per square centimeter of human skin. In addition, a large number of organisms reside deep in the dermis around the pilosebaceous follicles.

Frequently pathogenic bacteria are known to be able to penetrate the stratum corneum, the most superficial layer of the skin, protecting against exposure to topically applied antiseptics. And certain microbial species, such as Cutibacterium acnes, are known to reside deep within follicles, which are commonly plugged with sebum and dead skin cells. The majority of the topical antiseptic agents cannot penetrate deep into the dermal layers and hence cannot eradicate the residing microbes in these areas. For example, penetration of chlorhexidine into the skin is poor even following 30-minute exposure. While alcohols are sometimes used as penetration enhancers for topically applied agents, it has been demonstrated that dermal absorption is minimal. Iodine is capable of penetrating the dermis, but this is a slow time-dependent process and may take several hours to achieve levels considered bactericidal.

Therefore, Applicant has identified a need for an antiseptic that penetrates the dermis and treats sub-dermis layers. The present invention fulfills this need, among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

In one embodiment, the present invention is directed toward a topical antiseptic comprising a sebostatic agent, and an emollient, whose combined effects can enhance the dermal penetration and efficacy of active antiseptic ingredients. In another embodiment, the present invention is directed to a wipe comprising the above-mentioned antiseptic composition.

In one embodiment, the antiseptic composition comprises: (a) a sebostatic agent for reducing sebum production to prevent clogging of said pores; (b) a barrier-forming emollient for forming a protective barrier film to aid said dermis in maintaining moisture and to shield said dermis from the environment; and (c) an antiseptic agent.

In one embodiment, the composition comprises, a broad spectrum, fast-acting, persistent antiseptic; an alpha- or beta-hydroxy acid from about 0.01% to about 5% by weight to remove old skin cells and unclog skin pores; niacinamide from about 0.01% to about 5% by weight to regulate sebum production; and one or more long-chain fatty alcohols from about 0.01% to about 5% to enhance the skin's barrier function. In a more particular embodiment, the composition also comprises at least one of a humectant from about 0.01% to about 1% by weight, an essential oil from about 0.1% to about 1% by weight, and one or more surfactants from about 0.5% to about 5% by weight.

In another embodiment, the composition comprises a broad spectrum, fast-acting, persistent antiseptic; an alpha- or beta-hydroxy acid from about 0.01% to about 5% by weight to remove old skin cells and unclog skin pores; niacinamide from about 0.01% to about 5% by weight to regulate sebum production; and one or more long-chain fatty alcohols from about 0.01% to about 5% to enhance the skin's barrier function. In a more particular embodiment, the composition also comprises at least one of a humectant from about 0.01% to about 1% by weight, an essential oil from about 0.1% to about 1% by weight, and one or more surfactants from about 0.5% to about 5% by weight.

### DETAILED DESCRIPTION

In one embodiment, the present invention relates to an excipient combination to enhance efficacy of topical antiseptic agents by allowing it to penetrate deeper into the dermal layer. Any one or any combination of known antiseptic agents can be used. Preferably, the antisepsis should be broad, fast acting, and persistent. Suitable antiseptic agents include, for example, a short-chain alcohol, such as ethanol or isopropanol, a cationic surfactant, such as benzalkonium (BZK) chloride or chlorhexidine, or oxidizing agents, such as iodine complexes or peroxides. In a particular embodiment, the antiseptic agent comprises BZK, which Applicant has found to be particularly effective in treating problematic strains of acne as described below.

In one embodiment, the topical antiseptic comprises three main components, (1) an exfoliating agent, (2) a sebostatic agent, and (3) an emollient.

The exfoliating agent serves to remove old skin cells from the outermost layer of the epidermis, the stratum corneum. In doing so, exfoliation can aid in removing pathogens from skin surface and exposing microbes residing deeper in the skin and follicle to topically applied antiseptics. The exfoliating agent may be a chemical exfoliant or a physical (i.e., abrasive) exfoliant. Chemical exfoliation is commonly achieved through the application of organic acids, retinoic acid, or phenol to the skin. For the purpose of the intended application as a presurgical antiseptic preparation, only mild exfoliation should be achieved so as to not risk irritation or breaching the integrity of the skin. An organic acid would be best suited for this application, which could be one of a number of alpha-hydroxy acids, such as glycolic acid, lactic acid, citric acid, mandelic acid, tartaric acid, or malic acid; beta-hydroxy acids, such as salicylic acid or tropic acid; or poly-hydroxy acids such as gluconolactone or lactobionic acid. Additionally, inclusion of a buffering agent can ensure the antiseptic maintains a pH within a range that is both safe and effective. Typical pH of healthy skin is around 4.7, while the FDA cautions against use of exfoliating products with a pH less than 3.0 even when applied by trained professionals. In one embodiment of the invention, the exfoliative agent would be either an alpha- or beta-hydroxy acid, as they penetrate deeper than poly-hydroxy acids, at a concentration ranging from about 0.01% to about 5% by weight to achieve mild exfoliation along with an appropriate buffer at a concentration to achieve a pH between 3 and 6. In another embodiment, the exfoliation is achieved through mechanical action by applying the product to the skin with an abrasive cloth or wipe, and scrubbing to exfoliate the skin.

Sebum is a combination of oily substances produced by sebaceous glands that coat the skin preventing moisture loss. It is not uncommon for sebum and dead skin cells to build up and clog follicles, which plays a significant role in the development of acne. This can allow microbes, such as C. acnes, residing in the follicle to proliferate causing inflammation and making the area more susceptible to opportunistic pathogens. Additionally, sebum can serve as a fuel source for these microbes. Several topically applied agents have a sebostatic effect, preventing the overproduction of sebum, which includes antioxidants, niacinamide, retinoids, benzoyl peroxide, and cannabinoids. In addition to sebostasis, niacinamide is known to have anti-inflammatory properties as well as improves the epidermal barrier function. In one embodiment of the invention, the sebostatic agent would be niacinamide present at an amount from about 0.1% to about 5% by weight.

One of the primary functions of skin is to act as a barrier between living tissue and the external environment, preventing the invasion of foreign bodies, such as microbes that can cause infection, as well as preventing moisture loss. Sebum and the stratum corneum layer of the epidermis are crucial to this function, both of which may be reduced with application of this invention to enhance penetration of the active antiseptic agent. Therefore, it is important to enhance this barrier function. Any one of a number of potential ingredients can be used to accomplish this purpose. Emollients are semi-occlusive to occlusive substances that can form a protective layer over the skin. Examples of emollients include petrolatum, castor oil, long-chain fatty alcohols, cocoa butter, shea butter, silicone oils, and stearic acid. Other occlusive substances used in barrier creams include zinc oxide, talc, and kaolin. Humectants such as glycerin and aloe vera draw water toward the stratum corneum, maintaining a well-hydrated epidermis. While some substances may leave a greasy or sticky residue on the skin, long-chain fatty alcohols do not impart this quality and may reduce the sticky sensation associated with some of the cationic surfactant antiseptics. In one embodiment of the present invention, the selected barrier enhancing ingredient would be a combination of cetyl and stearyl alcohol, each present in an amount ranging from about 0.5% to about 4% by weight.

Addition of a humectant to the present invention can further improve the ability to maintain skin moisture, particularly if the active antiseptic ingredient is a short-chain alcohol which are known to dry out the skin. In one embodiment of the present invention, glycerin would be added to the formula present in an amount ranging from about 0.01% to about 5% by weight.

Inclusion of a fragrance can mask any unpleasant odors of other ingredients and make application of the invention a more pleasing experience for the user. Essential oils are a mixture of aromatic volatile oils extracted from plant sources that are commonly used to impart fragrance to topical products. In addition to a pleasant odor, many essential oils exhibit antimicrobial properties, with current evidence suggesting eucalyptus oil to possess the greatest antimicrobial activity. In one embodiment of the present invention, eucalyptus oil would be present in an amount from about 0.1% to about 1% by weight.

As a combination of hydrophobic and hydrophilic substances, a homogenous mixture will require the formula to be prepared as a colloid. Inclusion of a surfactant to the present invention can increase the stability of the prepared emulsion. Nonionic surfactants demonstrate the least cytotoxicity and risk of skin irritation in comparison to cationic, anionic, and zwitterionic surfactants. Classes of nonionic surfactants include fatty alcohol ethoxylates, alkylphenol ethoxylates, fatty acid ethoxylates, ethoxylated amines, fatty acid amides, terminally blocked ethoxylates, fatty acid esters of glycerol, fatty acid esters of sorbitol, fatty acid esters of sucrose, alkyl polyglucosides. In one embodiment of the present invention, one or more nonionic surfactants would be added to formula each present in an amount ranging from about 0.5% to about 5% by weight.

One particular embodiment of the composition is described below. It should be understood that the particular concentrations set forth below may vary +/− by 20% in one embodiment, 10% in another embodiment, and 5% in yet another embodiment. It should also be understood that, providing that the composition has at least one sebostatic agent, at least one barrier-forming emollient, and at least one antiseptic agent, the other listed components may be omitted or substituted depending on the application.

| | INCI/ | | | | | |
|---|---|---|---|---|---|---|
| | Common | Approved | | CAS | EILINCS | % |
| Trade name | Name | Suppliers | Function | No. | No. | W/W |
| SD Alcohol | Alcohol | Any USP | Solvent | 64-17-5 | 200-578-6 | 11.8 |
| 40B, 200 | Denat. | Grade | | | | |
| proof | | | | | | |
| Stearyl | Stearyl | Any USP | Barrier- | 112-92-5 | 204-017-6 | 0.005 |
| Alcohol USP | Alcohol | Grade | forming | | | |
| | | | emollient | | | |
| Cetyl Alcohol | Cetyl Alcohol | Any USP | Barrier- | 36653-82-4 | 204-017-6 | 0.01 |
| USP | | Grade | forming | | | |
| | | | emollient | | | |
| Glyceryl | Glyceryl | Any USP | Barrier- | 31566-31-1 | 250-705-4 | 0.005 |
| Stearate | Stearate | Grade | forming | | | |
| | | | emollient | | | |
| Polysorbate | Polysorbate | Any USP | Surfactant | 9005-64-5 | N/A | 0.05 |
| 20 USP | 20 | Grade | | | | |
| Purified | Water (Aqua) | USP Grade- | Solvent/ | 7732-18-5 | 231-791-2 | 85.095 |
| Water | | Local | Vehicle | | | |
| | | Source | | | | |
| Benzalkonium | Benzalkonium | Any USP | Antiseptic | 85409-22-9 | 85409-22-9 | 0.258 |
| Chloride 50% | Chloride | Grade | agent | | | |
| Aq. Solution | Water (Aqua) | | Solvent/ | 7732-18-5 | 231-791-2 | 1 |
| USP | | | Vehicle | | | |
| Niacinamide | Niacinamide | DSM | Sebostatic | 98-92-0 | 202-713-4 | 0.5 |
| PC | | Nutritional | agent | | | |
| | | Products | | | | |
| Glycerin, | Glycerin | Any USP | Humectant | 56-81-5 | 200-289-5 | 0.05 |
| Natural, USP | | Grade | | | | |
| Eucalyptol | Eucalyptus Oil | Any USP | Fragrance | 470-82-6 | 295-995-3 | 1 |
| Natural | | Grade | (Parfum) | | | |
| USP/FCC | | | | | | |
| PURASAL ® | Sodium | Corbion | pH Buffer | 867-56-1 | 212-762-3 | 0.01 |
| S/HQ 60 | Lactate | | | | | |
| Lactic Acid | Lactic Acid | Any USP | Exfoliating | 79-33-4 | 200-018-0 | 0.167 |
| (88% Aq. | | Grade | agent | | | |
| Solution) | Water (Aqua) | | Solvent/ | 7732-18-5 | 231-791-2 | 0.05 |
| | | | Vehicle | | | |

### Example 1

To produce 3000 g of antiseptic product, 0.150 g of stearyl alcohol, 0.30 g of cetyl alcohol, and 0.150 g of glyceryl stearate are combined in the 354 g of SD Alcohol 40B and mixed at 400 rpm at room temperature until liquid. Once the stearyl alcohol, cetyl alcohol, and glyceryl stearate have entirely melted, 1.5 g of polysorbate 20 is added to this phase A.

At the same time, 2614.86 g of water in a separate container is at room temperature, this is phase B. While continuously stirring, the phase A is slowly added to the phase B. Once completed, active antiseptic ingredient 7.74 g of benzalkonium, 15 g of niacinamide are mixed to phase B.

During this phase, the emulsion is continuously stirred. For phase C, the remaining components are added including, 1.5 g glycerin, 0.3 g eucalyptus oil, 3 g sodium L-lactate.

Maintain room temperature and mixing, for pH performance phase D, add 1.5 g of lactic acid.

### Example 2

To produce 100 mL of antiseptic product, 1.46 g each of stearyl alcohol, cetyl alcohol, and glyceryl stearate are combined in a single container and heated at 70 □C until liquid. At the same time, 15.2 mL of water, 1.95 g of polysorbate 20, and 70 mL of isopropanol are mixed in a separate sealed container and heated at 70 □C. Once the stearyl alcohol, cetyl alcohol, and glyceryl stearate have entirely melted, 0.5 mL of eucalyptus oil is added to this oil phase. While continuously stirring the oil phase, the aqueous phase is slowly added to the oil phase. Once completed, the emulsion is removed from heat and allowed to cool to ambient temperature. During this cooldown phase, the emulsion is continuously stirred, and the remaining components are added including, 0.044 g lactic acid, 0.05 g of glycerin, 0.5 g niacinamide and 0.75 g sodium lactate. A high shear mixer or homogenizer can be used at this stage to ensure a homogenous emulsion is achieved but is not required.

### Example 3

To produce 100 mL of antiseptic solution, 1.46 g each of stearyl alcohol, cetyl alcohol, and glycerol monostearate are combined in a single container and heated at 70° C. until liquid. At the same time, 70.2 mL of water, 1.95 g of polysorbate 20, and 15 mL of tertiary butanol are mixed in a separate sealed container and heated at 70° C. Once the stearyl alcohol, cetyl alcohol, and glycerol monostearate have entirely melted, 0.5 mL of eucalyptus oil is added to this oil phase. While continuously stirring the oil phase, the aqueous phase is slowly added to the oil phase. Once completed, the emulsion is removed from heat and allowed to cool to ambient temperature. During this cooldown phase, the emulsion is continuously stirred, and the remaining components are added including, 7.03 g lactic acid, 0.5 mL glycerin, 1.22 g niacinamide, and the active antiseptic ingredient, 0.13 g benzalkonium chloride. A high shear mixer or homogenizer can be used at this stage to ensure a homogenous emulsion is achieved but is not required.

### Example 4

To produce 100 mL of antiseptic solution, 1.46 g each of stearyl alcohol, cetyl alcohol, and glycerol monostearate are combined in a single container and heated at 70° C. until liquid. At the same time, 15.2 mL of water, 1.95 g of polysorbate 20, and 70 mL of isopropanol are mixed in a separate sealed container and heated at 70° C. Once the stearyl alcohol, cetyl alcohol, and glycerol monostearate have entirely melted, 0.5 mL of eucalyptus oil is added to this oil phase. While continuously stirring the oil phase, the aqueous phase is slowly added to the oil phase. Once completed, the emulsion is removed from heat and allowed to cool to ambient temperature. During this cooldown phase, the emulsion is continuously stirred, and the remaining components are added including, 7.03 g lactic acid, 0.5 mL glycerin, 1.22 g niacinamide and 0.75 g sodium lactate. A high shear mixer or homogenizer can be used at this stage to ensure a homogenous emulsion is achieved but is not required.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein and are intended to be within the spirit and scope of the invention. Accordingly, the foregoing description is by way of example only, and not limiting. The invention is limited only as defined in the following claims and equivalents thereto.

What is claimed is:

1. An antiseptic composition for penetrating and treating the dermis and/or pilosebaceous follicle of a mammal, said antiseptic composing comprising:
a sebostatic agent for reducing sebum production to prevent clogging of skin pores;
a barrier-forming emollient for forming a protective barrier film to aid said dermis in maintaining moisture and to shield said dermis from the environment; and
an antiseptic agent.

2. The composition according to claim 1, further comprising an exfoliative agent for exfoliating a portion of the stratum corneum of said mammal and unclogging said skin pores

3. The composition according to claim 2, wherein said exfoliative agent comprises a keratolytic agent

4. The composition according to claim 3, wherein the exfoliative agent is an organic acid.

5. The composition according to claim 4, wherein the organic acid is an alpha- or beta hydroxy acid.

6. The composition according to claim 5, wherein the organic acid is lactic acid in a concentration ranging from 0.01% to 5%.

7. The composition according to claim 1, wherein the sebostatic agent is niacinamide.

8. The composition according to claim 7, wherein the niacinamide present in an amount from about 0.1% to about 5% by weight.

9. The composition according to claim 1, wherein the emollient is comprised of a single or combination of long-chain fatty alcohols.

10. The composition according to claim 9, wherein the long-chain fatty alcohols including cetyl and stearyl alcohol present in an amount from about 0.01% to about 5% by weight.

11. The composition according to claim 1, further comprising a surfactant present in an amount from about 0.01% to about 5% by weight.

12. The composition according to claim 11, wherein said surfactant is one or more nonionic surfactants.

13. The composition according to claim 12, further comprising an essential oil for fragrance present in an amount from about 0.01% to about 1% by wight.

14. The composition according to claim 13, wherein said essential oil is eucalyptus oil.

15. The composition according to claim 14, further comprising an alcoholic solvent present in an amount from about 5% to about 70% by weight.

16. The composition according to claim 1, further comprising a humectant.

17. The composition according to claim 16, wherein said humectant is a glycerin in an amount from about 0.01% to about 5% by weight.

18. The composition according to claim 1, further comprising a buffering agent.

19. The composition according to claim 18, wherein said buffering agent is an organic acid salt.

20. The composition according to claim 19, wherein said organic acid salt is sodium lactate.

21. The composition according to claim 20, wherein sodium lactate is present in an amount necessary to achieve a pH between 3 and 6.

22. The composition according to claim 1, wherein the remaining volume comprises water.

23. The composition according to claim 1, wherein the composition is prepared as an oil-in-water emulsion.

24. A wipe comprising the composition of claim 2, wherein the wipe has an abrasive surface to act as said exfoliating agent.

25. The wipe of claim 24, wherein the wipe comprises an absorbent, non-woven material in which said composition is absorbed.
